(19) United States
(12) Patent Application Publication
(10) Pub. No.: US 2014/0221948 A1
(43) Pub. Date: Aug. 7, 2014

(54) HYGIENIC OR PERSONAL CARE ARTICLE HAVING A CONTENT OF COPPER OR COPPER IONS

(71) Applicant: Birgit Riesinger, Munster (DE)

(72) Inventor: Birgit Riesinger, Munster (DE)

(21) Appl. No.: 14/180,198

(22) Filed: Feb. 13, 2014

Related U.S. Application Data
(63) Continuation of application No. PCT/EP2012/066466, filed on Aug. 23, 2012.

(30) Foreign Application Priority Data
Aug. 23, 2011 (DE) 10 2011 052 941.1
Dec. 14, 2011 (DE) 10 2011 056 429.2

(51) Int. Cl. A61L 15/46; A61L 15/22; A61L 15/62; A61F 13/00

(52) U.S. Cl. CPC A61L 15/46; A61F 13/00068; A61F 13/00063; A61L 15/22; A61L 15/62; A61L 2300/102; A61L 2300/404
USPC 604/319; 424/638; 424/635; 424/489; 424/490

(57) ABSTRACT

The subject matter of the invention is a hygienic or health care article having a content of hydroactive polymers and a content of copper or copper ions.

### CROSS-REFERENCE TO RELATED APPLICATIONS

The present application claims priority from and is a continuation from PCT Application No. PCT/EP2012/066466, filed Aug. 23, 2012; which claims priority from German Patent Application No. DE 10 2011 052 941.1, filed Aug. 23, 2011 and DE 10 2011 056 429.2, filed Dec. 14, 2011, all herein incorporated by reference in their entireties.

### BACKGROUND

The present invention relates to a hygienic or care article having a content of copper or copper ions.

Such a hygienic or care article is particularly suitable for absorbing exudate from chronic wounds, as they occur in diabetes, leg ulcer and similar diseases, for example.

Such a hygienic or care article is also suitable for absorbing other fluids, in particular body fluids and excretions.

The term “exudate” denotes a wound fluid discharged via pathological processes of a wound. Just as blood is responsible for transporting nutrients and other messenger substances and thus supplying different parts of the body, the exudate functions in a similar manner for supplying the wound bed and the healing processes occurring in it. In order to fulfill these numerous functions, the exudate contains a broad spectrum of components, resulting in a specific weight that is slightly higher than that of water. In that, it differs also from transudate which has a clearly lower specific weight with a low cell and protein content. Besides providing nutrients for fibroblasts and epithelial cells, the exudate influences the different processes of wound healing temporally and spatially as a result of its high content of growth factors and cytokines The latter are formed above all by thrombocytes, keratinocytes, macrophages, and fibroblasts. They influence the motility, the migration and the proliferation of the various cells participating in wound healing. Thus, the migration of cells into the wound base and the supply of the newly formed granulation tissue due to angiogenesis are equally promoted. Wound cleaning is also promoted by the exudate. It contains various serine, cysteine and aspartate proteases as well as matrix metalloproteases which decompose tissues by their strictly regulated activity.

The components of the physiological exudate are in particular salts, glucose, cytokines and growth factors, plasma proteins, proteases (particularly matrix metalloproteases), granulocytes and macrophages.

If there is no clear progress in the wound healing course, according to the different phases of wound healing, within a few weeks, one speaks of a chronic wound. But here lasting exudate phases are considered a complication, and they are referred to as pathological exudation, which can contribute to the wound becoming chronic. The underlying causes are usually complex and they can definitely be of systemic nature. However, on the basis of the above explained importance of the exudate for wound healing, it is not surprising that complications during wound healing are reflected in a clearly changed composition and action of the exudate.

Infections play a large role, particularly in chronic, poorly-healing wounds, as frequently encountered in diabetics, for example. Recently, this problem has worsened because of pathogenic germs that are resistant to antibiotics. In particular multiresistant germs, such as vancomycin-resistant enterococci, methicillin-resistant Staphylococcus aureus, and multiresistant Pseudomonas aeruginosa, for example, often play a fatal role today in clinical practice.

Thus, in the USA, 90,000 diabetic patients with leg ulcer undergo amputations of limbs annually, because they cannot be treated in any other way owing to infectious complications caused by multiresistant germs.

In the case of incontinence articles, particularly diapers, there is, on the other hand, the risk that in particular vancomycin-resistant enterococci, which find ideal living conditions in such an article when it is in use, multiply and, for example, attack bruised skin spots of the person wearing the article. This is a real risk particularly since incontinence patients frequently are bedridden and suffer, for example, from bedsores (for example, in the buttocks area), where an infection that is extremely difficult to treat may develop if such resistant germs migrate into such areas. Moreover, in such patients, due to their sometimes long disease history, there is also the risk of the existence of vancoymycin-resistant enterococci in the digestive tract.

In other hygienic and/or wound care articles, such as sanitary napkins, tampons, incontinence articles, diapers, patient undersheets or incontinence sheets, stoma pouches, stoma pouch inserts, foot mats, surgery drapes, Redon bottles, handkerchiefs and/or hyperhidrosis articles, for example, there is a risk that antibiotic-resistant pathogenic germs spread and represent an infectious risk, owing to the often favorable growth conditions in said articles.

Copper has been used for centuries for disinfecting fluids, solids and fabrics. Copper is a transition metal and, as a weakly reactive heavy metal, it belongs to the noble metals.

Today, copper is used, for example, in water purification, as an algae-destroying agent, a fungicide, a nematocide, molluscicide as well as an antibacterial and a vegetation

protection agent because, in contrast to the low sensitivity of human tissue to copper, microorganisms react exceedingly sensitively to the copper.

To date, no microorganisms are known that are capable of withstanding constant exposure to copper, in contrast to antibiotics. The reason is probably that copper simultaneously attacks different cell-specific targets of the microorganism.

WO 2006/048879 describes a method for treating blisters and lesions in which a material containing water-insoluble copper compounds, in contact with a fluid, releases copper(I) ions, copper(II) ions or combinations thereof. WO 01/74166 also describes an antimicrobial and antiviral polymer material comprising copper ions. It is shown that fibers made of the polymer material comprising copper ions deploy in vitro antimicrobial and antiviral properties. However, both the method and the polymer material are not suitable for treating deep, exudative chronic and/or slow-healing wounds.

This is the case particularly since both the described method and also the polymer material entail a frequent change of the dressing of such wounds. However, this change of the dressing material interferes with the healing of the wound and it is traumatic particularly in the case of chronic wounds and often exceedingly painful for the patients.

In addition, the described polymer material or the material containing water-insoluble copper compounds does not match the body contour of the patient. Thus, wound care articles made of these materials are disadvantageous, particularly if they are to remain for a longer time on the wound, in order to prevent the just mentioned detriment to wound healing.

### SUMMARY OF THE INVENTION

Provided herein are systems, methods and compositions for a hygienic or care article is provided which comprises: a) a content of hydroactive polymers, and b) a content of copper or copper ions.

The methods, systems, and compositions are set forth in part in the description which follows, and in part will be obvious from the description, or can be learned by practice of the methods, compositions, and systems. The advantages of the methods, compositions, and systems will be realized and attained by means of the elements and combinations particularly pointed out in the appended claims. It is to be understood that both the foregoing general description and the following detailed description are exemplary and explanatory only and are not restrictive of the methods, apparatuses, and systems, as claimed.

### DETAILED DESCRIPTION OF THE INVENTION

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

The aim of the present invention is to provide a hygienic or care article which does not have some of the above-mentioned disadvantages. This aim is achieved by means of a hygienic or care article according to the present main claim; the dependent claims indicate preferred embodiments.

Accordingly, a hygienic or care article is provided which comprises:

a) a content of hydroactive polymers, and

b) a content of copper or copper ions.

The mentioned hygienic or care article is preferably an article selected from the group consisting of wound care articles, sanitary napkins, incontinence articles, diapers, patient undersheets or incontinence sheets, stoma pouches, stoma pouch inserts, foot mats, surgery drapes, Redon bottles, handkerchiefs and/or hyperhidrosis articles. It can also be a care product, such as, for example, an ointment, a pasty preparation, a gel or the like.

The term “wound care article” below denotes in particular a wound dressing, preferably a flat dressing or a wound care cloth, which is intended for the sectors of human medicine, dental medicine and/or veterinary medicine.

Said wound care article can preferably be in the form of a swab, a wound cloth, a wound dressing, a wound compress, a wound pad, a resilient compress if necessary, a bandage, a band-aid or a sock.

In particular, the term “wound care article” can also be understood to refer to an assembly of different products which are arranged in a given arrangement on the wound to be treated. This assembly can form a physical unit, for example, if the different products are combined in a common shell or—optionally without a shell—connected adhesively to one another. However, this assembly can also be in the form of a kit, wherein the different products are arranged optionally using a compress, an adhesive band, an adhesive strip, or a band-aid in the given arrangement on the wound to be treated.

The term “patient undersheet or incontinence sheet” denotes products that are used in the care or hospital field as undersheets for patients or persons in need of care, and that are intended to absorb outflowing fluids.

Copper is found in enzymes participating in the energy metabolism and it is important for the formation of the red blood pigment (hemoglobin) as well as for the functioning of the cytochromes in the mitochondria. Copper is also important for the uptake of iron from food.

Therefore, in the case of a copper deficiency, symptoms of iron deficiency and functional impairments of the corresponding enzymes occur.

Not lastly, owing to the overwhelming success of the antibiotics introduced at the end of the 1930s, the interest in using copper for hygienic measures abated.

As the above-mentioned multi-resistant germs appeared, the interest in the use of copper for hygienic measures also increased again.

Thus, in a promising test of the Asklepios Clinic Wandsbek in Hamburg, an entire hospital station was fitted with door knobs, door panels and light switches made of copper. The underlying reason was that microorganisms are not transmitted only from hand to hand, but in many cases also by touching handles and switches.

Copper highly efficiently deactivates or kills a broad spectrum of viruses and microorganisms. Here, copper or copper ions act on different species.

1) Because they represent highly redox-active metal ions, they promote the peroxidation of membrane lipids and as a result damage the cell walls of the microorganism. As a result of this property, they displace, in addition, the essential metals from their original binding centers, thus interfering with the metabolism of the microorganism.

2) They damage the genetic material of the microorganism by binding to DNA and thus changing its conformation.

3) They damage the proteins of the microorganisms; in particular they cause the degradation of sulfhydryl groups and the oxidation of certain amino acid residues.

4) They interfere with oxidative phosphorylation and upset the osmotic balance.

In addition copper or copper ions influence the different biological defense mechanisms of the microorganism in such a manner that there is practically no remaining resistance against antimicrobial active ingredients.

Thus, the hygienic or care article according to the invention has the advantage that, due to copper or copper ions, it possesses both antimicrobial properties and also antiviral properties, and simultaneously—due to the hydroactive polymers—it adapts to the body contour of the user in an amorphous manner. This considerably increases the wearing comfort, particularly in the case of slow-healing, exudative, chronic and/or deeper wounds.

Besides copper, silver is also known for its antimicrobial properties. However, in contrast to copper, silver has the disadvantage that silver absorption through the skin leads to the accumulation of silver in the organism.

Besides argyria, an irreversible slate-gray discoloration of the skin and mucosal membranes, silver accumulation can lead to taste disorders, insensitivity to odors as well as cerebral seizures. Silver is also deposited in vessels and inner organs, such as the liver, the kidneys and the spleen. In this context, chronic upper abdominal pains and vertigo have been described. For this reason, in 1999, the FDA in the USA considered drugs for over-the-counter sale that contain silver salts or colloidal silver unsafe both for internal and external use, and a separate official management practice was created.

Thus, the hygienic or care article according to the invention has the advantage that, due to the copper or copper ions, it has both antimicrobial properties and also antiviral properties and at the same time—due to the hydroactive polymers—it adapts itself to the body contour of the user. This considerably increases the wearing comfort particularly in the case of slow-healing, exudative, chronic and/or deeper wounds.

An additional advantage of a hygienic or care article according to the invention, in particular a wound care article, is that said article can remain on the wound for a longer time than conventional wound dressings, due to the swelling capacity of the hydroactive polymers.

In principle it is precisely when the degree of colonization of a wound is not known exactly that a frequent change of dressing is required, in order to be able to intervene rapidly in the case of an infection. The wound care article according to the invention makes it possible to dispense with such a frequent change, because it enables the control of infections and thus meets the need for increasing the wound resting phases, which is necessary according to experts in the field of modern wound care, particularly in the case of diabetes-specific chronic wounds.

Since copper is a trace element that is required for life and since it stabilizes the layers of skin, it is safe for the skin and for use on wounds. Moreover, more recent research has shown that copper or copper ions even promote wound healing by inducing the growth of capillaries and the regeneration of the epidermis, and by initiating or stimulating the formation of collagen, fibronectin, elastin, integrin, and other key proteins.

Below, the term “antimicrobial properties” denotes a substance that reduces the multiplication capacity or infectivity of microorganisms, or even kills or inactivates microorganisms, that is to say acts as a bactericide, for example.

Below, the term “microorganisms” denotes microscopically small organisms. Examples of microorganisms are bacteria, fungi, microscopic algae, and protozoa.

Below, the term “antiviral properties” denotes substances that are directed against viruses and the diseases caused by them. In general, they inhibit the multiplication of virus particles in the body and thus have a virostatic effect. Antiviral substances frequently intervene in enzymatic processes that are required for the multiplication of the virus particles and at the same time they are specific for the virus in question. An additional mechanism of action of antiviral substances involves prevention of the accumulation of the virus particles on the cell and their penetration into the cell. In addition, the maturing of new virus particles or their export can be prevented, so that no additional cells can be attacked and the infection cannot spread further.

It is preferable for the copper ions to be copper cations. In an embodiment, the copper ions are copper(I) and/or copper(II) ions, that is to say monovalent or bivalent copper ions. In an additional embodiment, the copper ions are bound copper ions. The term “bound copper ions” below means that the copper ions are first present as a combination with another substance, for example, as a salt (referred to below as copper salts) and thus they carry no external charge. Examples of such bound copper ions are copper sulfate, copper oxychloride, copper chloride and/or copper hydroxide.

In an embodiment, the bound copper ions become detached from the composition by contact with wound fluid or exudate and then they deploy their antimicrobial and antiviral properties. However, it is naturally also possible for the copper ions to deploy their antimicrobial and antiviral properties in the form of a combination with another substance.

The bound copper ions or elemental copper have the advantage that, depending on the use, the copper ions can be integrated in a more simple manner in the hygienic or care article according to the invention, since they are less reactive.

The bound copper ions are preferably copper oxide. The term “copper oxide” below denotes copper(I) oxide, copper(II) oxide or a mixture of the two copper oxides.

It is preferable for the copper salt, preferably the copper oxide, to be in the form of particles having a size between ≧0.5 μm and ≦20 μm. The size is particularly preferably between ≧1 μm and ≦5 μm.

In an embodiment, the copper or the copper ions are present as fibers, in powder form, in granulate form, in wire form, as a colloid, and/or as cuttings.

The copper ions are preferably present as fibers that are impregnated with ≧0.1% to ≦15% copper ions. Fallback positions are described further below in this application. Alternatively, it is also possible to provide fibers that are impregnated with elemental copper or into which wires, braids or filaments made from elemental copper are incorporated. Alternatively, fibers can also be used that consist entirely of wires, braids or filaments made of elemental copper.

Below, the term “impregnated” means that fibers made, for example, of cellulose, nylon, polyester, acrylic or polyethylene and polypropylene are mixed during their preparation, i.e., before the extrusion, with ≧0.1% to ≦15% copper ions. Fallback positions are described further below in this application.

For example, the copper ions can be present in the form of a colloid. The term “colloid” below denotes copper ion particles that are finely distributed in another medium (solid, gas or liquid), namely the dispersion medium. The size of the individual colloid is typically between 1 nm and 1 μm. If the colloid particles are mobile (for example, in a liquid medium), then the colloids usually exhibit Brownian motion.

Said colloid can be applied, for example, in the form of an ointment on the hygienic or care article comprising a content of hydroactive polymers.

Below, the term “hydroactive polymers” denotes polymers that have moisture-binding properties. They include among others:
Agar
Alginate
Carrageenan
Locust bean gum
Guar gum
Tragaganth gum
Gum arabic
Xanthan gum
Karaya gum
Tara gum
Gellan gum
Pectin
Chitosan
Hyaluronic acid
Modified starches
Celluloses, modified celluloses and cellulose ethers, such as, for example, carboxymethylcellulose, hydroxypropylcellulose, hydroxypropylmethylcellulose, methylcellulose and methylethylcellulose, as well as
superabsorbent polymers (SAP).

The mentioned hydroactive polymers absorb wound exudate and in the process they also form a moist surface. In some cases, they assume a gel like form. The moist surface or the gel form contributes to reducing the tendency of the wound care article to adhere to the wound, so that the article can be removed without trauma and painlessly after its use. Due to the gel form, the wound care article has a cooling and thus pain relieving effect. In addition, the gel form allows the development of a moist climate that promotes wound healing.

The hydroactive polymers are particularly preferably alginates. The latter are extracted from brown algae and woven to a fibrous web. Chemically they are polysaccharides, in particular calcium and/or sodium salts of alginic acids. Alginates are capable of absorbing up to 20 times their own weight of fluid; in the process the wound exudate is incorporated in the cavities. The Ca²⁺ ions contained in the alginate matrix are replaced with the Na⁺ ions from the exudate until the Na-ion saturation degree in the alginate has been reached. In the process, a swelling of the wound dressing occurs, and the alginate fibers are converted to a gel mass by swelling of the fibers.

Modified celluloses are preferably derivatives of cellulose, preferably sulfoalkylated cellulose and its derivatives, in particular sulfonated or sulfated cellulose derivatives, preferably cellulose ethyl sulfonates, carboxylated cellulose, preferably carboxymethylcellulose, carboxyethylcellulose and/or carboxypropylcellulose, more complex cellulose derivatives, such as sulfoethylcarboxymethylcellulose, carboxymethylhydroxyethylcellulose, hydroxypropylmethylcellulose, and amidated cellulose derivatives, such as carboxymethylcellulose amide or carboxypropylcellulose amide. Carboxymethylcellulose is in particular in the form of sodium carboxymethylcellulose, and it is available commercially under the name “Hydrofiber.” In hygienic and wound products, the fibers are converted into a flat matrix. Due to the absorption of fluid from the wound exudate, the fibers are gradually converted into a gel pad which retains the fluid and no longer releases it. Here, the structure of the fibers is such that the wound exudate is absorbed only in the vertical direction. This means that, as long as there is sufficient capacity, the exudate does not flow over the wound margin. In this way, wound margin maceration can be effectively prevented.

The hydroactive polymers are particularly preferably superabsorbent polymers. Superabsorbent polymers (SAP) are plastics capable of absorbing a multiple of their own weight to 1000 times their weight—of fluids by suction. Chemically, they are a copolymer of acrylic acid (propenoic acid, C₃H₄O₂) and sodium acrylate (sodium salt of acrylic acid, NaC₃H₃O₂), wherein the ratio of the two monomers to each other can vary. In addition, a so-called core cross linker (CXL) is added to the monomer solution, which binds the formed long-chain polymer molecules to one another by chemical bridges in some sites (it “crosslinks”). As a result of these bridges, the polymer becomes water insoluble. When water or aqueous salt solutions penetrate into the polymer particles, the latter swell and they stretch this network on the molecular level, so that, the water can no longer escape without assistance.

Alternatively, the superabsorbents can be selected from methacrylic acid, polyvinyl alcohol-maleic acid anhydride copolymers, polysaccharide-maleic acid anhydride copolymers, maleic acid derivatives, acrylamidopropanesulfonic acid copolymers, starch-acrylonitrile graft polymers, gelatinized starch derivatives, alkyl- or hydroxyalkylcellulose, carboxymethylcellulose, starch-acrylic acid graft polymers, vinyl acetate-acrylic acid ester copolymers, acrylonitrile or acrylamide copolymers. Similarly, the mentioned hydroactive polymers can also be hydrogel nanoparticles comprising hydroxy terminated methacrylate monomers, such as 2-hydroxyethyl methacrylate (HEMA) and/or 2-hydroxypropyl methacrylate (HPMA), which are marketed as Altrazeal, for example.

In the hygienic or care articles according to the invention, the superabsorbent polymers can be in the form of a granulate, a powder, a bulk preparation, a pellet, a foam, in the form of fibers, a fiber knit, mat or nonwoven and/or a fiber wadding.

The superabsorbent particles can be in powder or granulate form with a particle size between 100 μm and approximately 1000 μm.

The mentioned hygienic or care article is preferably a wound dressing containing superabsorbent polymers whose structure corresponds approximately to the commercially available products “sorbion sachet,” “Tenderwet,” “Zetuvit,” “Drymax,” “Vliwasorb” and/or “Curea P.”

It is preferable here to provide that the wound care article functions as a rinsing body. For this purpose, it is provided that the wound care article is soaked prior to the application to the wound with a physiological solution (for example, 0.9% sodium chloride, Ringer solution or the like) or that it is prepackaged after having been soaked correspondingly. Such a wound care article continuously releases fluid to the wound while it is in use, it rinses the wound, and it absorbs exudate, cell debris, necrotic components, bacteria, debris and the like.

As the applicant has shown in previous patent applications, superabsorbent polymers are capable of absorbing and binding large quantities of exudate. In this manner, they reduce the content of pathological exudate in the wound and thus promote wound healing.

However, the mentioned hydroactive polymers—particularly the superabsorbent polymers—bind not only fluids, but also bacteria, proteins, and other biomolecules.

This effect is complemented perfectly by the antimicrobial and antiviral effect of the copper or copper ions. This is the case, since studies have shown that copper or copper ions deploy their antimicrobial and antiviral properties within the briefest time. As a result particularly if the microorganisms are bacteria—a release of endotoxins and bacterial pathogenicity factors (in particular bacterial hemolysin and leukocidin) can occur. The latter in turn can induce inflammations, allergies, shock (particularly anaphylactic shock and/or toxic shock syndrome), and fever (Herxheimer reaction). These conditions occur particularly when copper oxide is applied topically, for example, in the form of a hygiene or a care article furnished in the mentioned manner, for example, of a wound dressing.

A hygienic or care article according to the invention, comprising a content of superabsorbent polymers and a content of copper or copper ions, preferably copper oxide, consequently not only contributes to reducing the number of pathogens in the wound by the absorption of microorganisms, particularly bacteria, by means of SAP and/or by lysis of bacteria; but also actively absorbs the lysates produced, and in particular the endotoxins contained therein.

The latter feature also contributes to reducing the stress on the immune system, which otherwise would have to process and dispose of the endotoxins formed.

A synergistic effect can also be described in relation to the degradation of persistent biofilms. Thus, the superabsorbent polymers are capable of drying out biofilms, so that cracks form, through which copper or copper ions can exert their bactericidal action. Copper is known for its biofilm-degrading action, which is important particularly in the construction of pipelines. This action is moreover complemented by the bacteriostatic and bactericidal properties of superabsorbent polymers as well as by their potential for modulating tissue-damaging proteases (binding, inactivation, and removal).

An additional advantage is achieved with hygiene and care articles according to the invention that are not in direct contact with the wound, such as patient undersheets or incontinence sheets, for example. The solubility of copper and copper salts, such as copper(I) oxide and copper(II) oxide, is strongly pH dependent. In the slightly acidic range, as generated by the presence of superabsorbent polymers based on polyacrylates, the release of copper or copper ions from the product is increased, and they are then available for the inactivation of the bacteria and microorganisms contained in the absorbed body fluids (intended burst). Furthermore, it can be guaranteed that, after the fluid absorption, due to an increase of the pH, the solubility of the copper or of the copper and the copper salts decreases again and/or the acrylate anions, whose presence is increased, bind the released copper ions to the cellulose-SAP matrix.

An additional advantage of a hygienic or care article according to the invention, in particular a wound care article, is that it not only can be used for treating wounds infected with antibiotic-resistant germs, but it can also be used in cases where, for other reasons, antibiotic therapy is not indicated. This applies, for example, to
pregnant women, who as a rule should be treated only with a very limited antibiotic spectrum (in particular erythromycin)
persons suffering from an antibiotic allergy, and
persons with liver damage or systemic defects, who are at risk for a metabolic intoxication in the case of an antibiotic therapy.

An additional advantage of the hygienic or care article according to the invention is that it is particularly suitable for treating burn wounds which, on the one hand, produce strong exudation (which makes the use of SAP appropriate), but which, on the other hand, end up being barely supplied with blood or not at all, which makes a systemic antibiotic therapy difficult. However, burn wounds are exceedingly susceptible to multiresistant germs, so that in this case a treatment with copper or copper ions represents a new promising option.

An additional advantage of the hygienic or care article according to the invention is the common occurrence of synergistic reduction of odor formation due to infection, which is often encountered in the case of chronic wounds. Due to the activity of the copper or of the copper ions, the metabolic activity of the bacteria, which is responsible for odor formation, and which releases in particular butyric acid, is reduced. At the same time, the superabsorbent polymers absorb both already formed odorous substances and also exudate and water from the wound and bind them. As a result of the drying of the wound, the growth conditions for the odor-inducing bacteria are worsened, leading to a further reduction of odors.

The hygienic or care article according to the invention can be particularly advantageous if elemental copper or copper(I) and copper(II) ions are mixed with hydroactive polymers in the form of superabsorbent polymers. Indeed, a colloid can form from the wound fluid or exudate absorbed by the SAP. In the colloid, the copper ions made available, for example, in the form of a fiber impregnation of the wound and/or a care article, particularly if they constitute a mixture of copper(I) and copper(II) ions, can alternate due to a redox reaction between the more stable copper(II) ion and the copper(I) ion which has a stronger antiviral action. This has the advantage that the hygienic or care article according to the invention can be stored longer due to the copper(II) ions present.

The advantageous formation of a colloid can occur even if the copper ions previously were in the form of bound ions, for example, as copper(I) and/or copper(II) oxide, or even in elemental form. The reason appears to have to do with the composition of the wound exudate in which the copper ions dissolve more readily from the copper oxide than in pure water.

This advantageous formation of a colloid is surprising, because SAPs are known to bind exudate sufficiently effectively so that in fact no fluid should be present for forming the colloid. However, it is evident that the gel formation of the superabsorbent polymer is sufficient to allow the formation of a colloid, and thus the advantageous transfer of the copper or copper ions. This implicit and surprising type of colloid formation, in comparison to intentionally added colloids, for example, in the form of ointments that are applied onto the hygienic or care article according to the invention, has the advantage that the manufacture can occur much more efficiently and cost effectively. Since elemental copper or copper ions, for example, bound as copper oxide, can be mixed simply with the superabsorbent polymers during the manufacture of the hygienic or care article according to the invention, or a spatial proximity of the two contents can be ensured in another manner. For example, fibers made of superabsorbent polymers can simply be impregnated with elemental copper or copper ions, for example, in the form of copper oxide. However, in the same manner, the entire hygienic or care article can also be manufactured from a fiber mixture, wherein some fibers comprise superabsorbent polymers and some elemental copper or copper ions.

Furthermore, it is provided according to the invention that, in the case of superabsorbent polymers, the polymers are in powder or granulate form.

Superabsorbent polymers in this form of presentation have numerous advantages, which are known, for example, from WO0152780 and WO03094813 of the applicant of the present invention.

Alternatively, the superabsorbent polymers can be in fiber, yarn, wadding, nonwoven web, knit, airlaid, nonwoven and/or woven form.

The term “airlaid” denotes a special nonwoven web made of cellulose and polyolefin fibers, in which superabsorbent polymers are optionally embedded.

It is particularly preferable for the superabsorbent polymers to be in the form of an absorbent body comprising a nonwoven web that contains cellulose fibers.

The absorbent body can comprise a substantially flat absorbent body made of absorbent material, which consists of a superabsorbent nonwoven web with superabsorbent polymers distributed therein. They can be in the form of a granulate, a powder, a bulk preparation, a pellet, a foam, in the form of fibers, a fiber knit, mat or non-woven, or a fiber wadding.

Here, the absorbent body comprises at least one material which is selected from the group containing a mat, in particular from an airlaid material made of said yarns or fibers made from superabsorbent polymers with incorporated superabsorbent polymers, and/or a loose filling made of superabsorbent polymers. Said airlaid mat can preferably comprise a substantially flat material section made of absorbent material, which consists, for example, of an absorbent nonwoven web made of the mentioned fibers with superabsorbent polymers distributed therein.

This absorbent body can correspond to the absorbing insert which is contained in a wound dressing of the applicant of the present invention, as disclosed in WO03094813, WO2007051599 and WO0152780, for example. The disclosure content of the mentioned patents is herein incorporated by reference in their entireties. In another embodiment, the absorbent body can also form a core comprising—optionally tuft-shaped—fibers or yarns made of superabsorbent polymers as well as superabsorbent polymers in granulate form, wherein the granulates are glued or welded to the fibers or yarns at several heights, and the granulates are distributed over more than 50% of the entire construction height of at least one section of the core, wherein there are mixed areas of granulate and fibers. The proportion by weight of the superabsorbent polymers can here preferably be in the range between ≧10 and ≦100 wt %.

Here, the following values are particularly preferable: 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100 wt %.

Similar constructions are known from conventional incontinence materials, and, like sanitary napkins, are known for their padding properties. Around said core, a shell can be arranged, which in some areas is arranged with overlap, and which covers, for example, a gluing seam or a part thereof.

It is particularly preferable for the absorbent body to comprise a nonwoven web, preferably a nonwoven or airlaid material, which consists of superabsorbent fibers (“SAF,” preferably polyacrylates) or contains said superabsorbent fibers as a component. The fibers can be mixed, for example, with fluff pulp (cellulose) or with polyester fibers. Alternatively or additionally a layer construction can be provided.

In another embodiment, the absorbent body can also contain at least one flat layer comprising fibers or yarns made of superabsorbent polymers, to which superabsorbent polymers in granulate form are glued. The result, in a preferred embodiment, is a construction of the body, which comprises at least two layers, wherein at least one cover layer underlies a layer comprising superabsorbent polymers. Optionally, a second, flanking cover layer can be provided.

Here, in the plane, there are no mixtures of fibers and superabsorbent polymers; instead, there are only the two materials secured adjacently. The optionally provided several layers can here also be physically compacted together in a preferred embodiment, by rolling, compressing, calandering and similar methods.

Moreover, a material can also be provided in which it is not the superabsorbent polymers that are not introduced into a cellulose matrix, but an amorphous material that is introduced, which comprises superabsorbent polymers and which is embedded, for example, between two layers which consist of a nonwoven web comprising cellulose or another material.

The use of superabsorbent polymers in fiber or yarn form proposed here comprises several advantages in comparison to particulate superabsorbent polymers:

i) Thus, said fibers or yarns have a wick effect. In this manner, when in contact with a fluid, they are capable of absorbing and binding this fluid much more rapidly than particulate superabsorbent polymers.

ii) In addition, the fluid flows are capable of channeling or directing. In this manner it is possible, for example, to prevent wound margin maceration.

iii) Unlike particulate superabsorbent polymers, they can be processed to the form of nonwoven webs, fabrics or the like. In this manner it is possible, for example, for the shell of a wound dressing to be manufactured from these fibers or yarns, and the superabsorbent properties can thus be brought much closer to the wound base in this manner.

iv) Since a separate carrier material can in many cases be dispensed with, the content of superabsorbent materials in a hygienic or care article, particularly in a wound care article, can be increased considerably; in the extreme case, it can even amount to a proportion by weight of 100%.

v) Said fibers or yarns, or the products produced therefrom, have a much higher softness and a lower abrasiveness than the corresponding particulate superabsorbent polymers.

vi) Said fibers or yarns can be processed to a structure without requiring a glue or a welding process, as is required in the case of particulate superabsorbent polymers. This entails considerable advantages both with regard to the purity of the product and with regard to the pharmacology and any allergenicity.

vii) In contrast to particulate superabsorbent polymers, the dimensioning of said fibers or yarns can be controlled and checked much more precisely, which, on the one hand, leads to preventing dust, which is frequently produced when using particulate superabsorbent polymers, and, on the other hand, considerably increases the product quality (homogeneity and reproducibility).

viii) Due to the absence of dust formation, it is also possible optionally to dispense with the use of a separate shell.

ix) In airlaid materials known from the prior art, said yarns or fibers can, in airlaid materials known from the state of the art which contain particulate superabsorbent polymers, replace the carrier fibers of the airlaid material, in order to increase the content of superabsorbent materials in a hygienic or care article, particularly in a wound care article, and thus the entire absorption capacity.

Any two- or three-dimensional arrangement of the fibers or yarns is conceivable here. Thus, the fibers or yarns can be arranged with orientation or without orientation (tangled), in several layers or otherwise.

Superabsorbent fibers made of polyacrylates are offered and marketed, for example, by the company Technical Absorbents under the commercial name “Oasis Super Absorbent Fibre.” Like all superabsorbent polyacrylates, they have a very high fluid absorption capacity. These fibers can be, for example, in the form of a nonwoven web, an airlaid material, a fabric, an airlaid material and/or a nonwoven. Such fibers are known from DE 69807337, for example.

Moreover, it is preferable for the superabsorbent polymers that are in fiber form to be at least partially in the form of a wadding, a nonwoven web, an airlaid material and/or a nonwoven.

For this purpose, it is preferable to use fibers having mean lengths of 5-50 mm. The manufacturing occurs by known methods, such as carding or the airlaid procedure, for example. Here, the superabsorbent fibers can be the sole component of the given material. However, it is preferable for the hygienic or care article to comprise in addition a content of supporting fibers, which, even in the moist state, ensure the integrity of the wound dressing, wherein said fibers are selected from the group containing

a) cellulose fibers,

b) viscose fibers,

c) hollow fibers,

d) alginate fibers, and/or

e) polyester, polyolefin, polyurethane, polyvinyl alcohol or polymaid fibers or mixtures or copolymers thereof.

For this purpose, in particular so-called bonding points can also contribute, as produced by the locally specified induction of so-called “hydrogen bonding” bonds.

In this manner, it is possible to precisely control the absorption properties of the material as well as other properties, such as the extensibility, the tensile strength, the behavior when drenched, and the like, for example.

Here, it is possible to provide in particular a fabric, wherein the warp threads are made of superabsorbent yarns, for example, while the weft threads consist of other yarns which contain supporting fibers according to the above list, for example. Such a fabric has oriented hydroactive properties, i.e., it takes up fluid in one direction and moves it on in the other.

In an embodiment, the hygienic or care article according to the invention comprises at least one component from the group including active charcoal and silver ions.

This has the advantage that the antimicrobial or odor-binding properties of the copper or copper ions are further promoted.

In an embodiment, the hygienic or care article according to the invention comprises a material section made of superabsorbent polymers and a shell arranged at least in some sections around this material section.

It is preferable for an optionally provided shell to consist at least partially of a hydrophobic material, for example, polypropylene, or another hydrophobized natural material, such as cotton. The hydrophobic properties of the shell prevent sticking to the wound surface and they contribute to the wound exudate reaching the interior of the shell more rapidly.

The shell can also be produced from another plastic, particularly a polyurethane or polyurethane film or from synthetic spider silk film.

In addition, it is preferable to provide that the shell of a hygienic or care article according to the invention comprises at least in some sections an adhesive coating, in particular preferably on the side facing away from the wound, by means of which it can be attached in the wound area for example, with a bandage.

Moreover, it is also possible to provide that the shell comprises an area that extends over the wound itself and that is provided with adhesive strips for attachment.

It is preferable for the copper or the copper ions and/or the superabsorbent polymers to be arranged within the shell; however, they can also be arranged in or on the shell or be a component thereof. This is particularly possible if a flat shell material in the form of a web is used which contains copper or copper ions at least in some sections. For this purpose, fibers can be used that are provided with or impregnated with copper or copper ions, or are made directly from copper or copper ions (as described elsewhere herein), in order to manufacture therefrom —optionally with other fibers, such as woven synthetic fibers or synthetic fibers assembled as in the form of a nonwoven web, such as polypropylene or polyethylene fibers, but also cotton, silk or viscose—yarns, nonwoven webs, woven fabrics or knitted fabrics, from which said flat shell material can then be manufactured. The latter can then preferably be used to enclose an absorbent body comprising superabsorbent material. Copper or copper ions can, however, also be introduced into one of the nonwoven materials described in this application, which contain superabsorbent polymers.

Copper or the copper ions can also be put in or on a matrix material. This can be achieved, for example, by surface coating by PECVD (Plasma Enhanced Chemical Vapor

Deposition), CVD (chemical vapor deposition) or dip coating, or, on the other hand, by introducing copper-containing or non copper-containing fibers or yarns into the material by coextrusion or simultaneous spinning or felting.

The matrix material preferably comprises a plastic or plastic fiber content. Said matrix material can be, for example, polyester, PLA polyester, nylon, polypropylene, polyethylene or latex.

The copper content can then be in the range between ≧0.1 and ≦20 wt %, preferably between ≧1 and ≦10 wt %, particularly preferably between ≧2 and ≦5 wt %. Most particularly preferable values are in particular 1, 2, 3, 4, 5, 6, 7, 8, 9, 10, 11, 12, 13, 14, 15, 16, 17, 18, 19 or 20 wt %.

It is particularly preferable to introduce the copper or copper oxide into a nonwoven web made of plastic fibers. For this purpose, a layer made of a hydrophobic spunbonded fabric is produced, which comprises a plastic or plastic fiber content, made, for example, of polypropylene fibers that are impregnated with copper oxide particles (preferred copper oxide content as above, particularly preferably ≧2 and ≦5 wt %).

Said spunbonded fabric can have a density between ≧2 and ≦100 g/m², preferably between ≧5 and ≦50 g/m², particularly preferably between ≧10 and ≧30 g/m², even more preferably between ≧15 and ≦20 g/m² or between ≧23 and ≦28 g/m².

Since the plastic or plastic fibers are commonly apolar and thus hydrophobic, said spunbonded fabric can preferably comprise a hydrophilizing coating (so-called “avivage substance”).

Alternatively, copper or copper oxide can be introduced into a highly absorbent needle punched nonwoven. For this purpose, modified cellulose fiber (viscose/rayon) at a density of 1-2 denier (preferably: 1.3-1.5 denier) and having a length of 20-50 mm (preferably 35-40 mm), for example, is used, and it functions as absorbent layer. To this nonwoven web, ≧2 and ≦5 wt % copper-coated cellulose fibers are admixed during the manufacturing process. This absorbent layer can furthermore receive, as described elsewhere herein, admixtures of superabsorbent fibers, for example, in granulate or fiber form.

It is particularly preferable to use a combination of a spunbonded nonwoven as described above, made, for example, of polypropylene fibers impregnated with copper oxide particles, and an absorbent layer, as described elsewhere herein, comprising superabsorbent polymers, wherein both layers can be connected to one another, preferably without glue. This can occur, for example, by means of known methods such as ultrasound welding or thermal binding, for example.

It is preferable for the shell of the care or hygienic article to comprise a hydrophobic material or for the shell material to have a hydrophobic finishing. The hydrophobic properties of the shell prevent sticking to the wound surface and they contribute to the wound exudate particles being able to reach the interior of the shell more rapidly.

Here, it is possible to provide that the shell comprises at least in some sections a resilient material, such as fibers made of Lycra or elastane, for example. This also ensures that the material section, in the case of fluid absorption, can increase in volume and is not restricted by the shell.

Said shell encloses the absorbent body, forms a barrier against solid excretions, and allows the passage of other exiting substances toward the material section made of absorbent material which is arranged inside the shell. The shell is preferably closed off at least partially by a seam.

The size of the pores or meshes of the shell is preferably 0.05 mm to 1.0 mm, advantageously 0.20 mm to 0.50 mm. In principle, it is possible to provide here that the mean pore size ends up being smaller than the mean size of the particles containing hydroactive polymers.

Moreover, it is possible to provide preferably for the pores or meshes to be delimited by material sections, which, in section through the shell, are roughly arc-shaped and point outward with their arc ridges. This applies both to nonwoven materials and also to film materials.

Absorbent bodies of the mentioned type—but without copper—are disclosed, for example, in WO03094813 and WO2007051599 of the applicant of the present invention.

The material of the shell can be structured in such a manner that the shell has a rough inner surface and a smooth outer surface. It is preferable for the rough inner surface of the shell to be formed by funnel-shaped perforations which narrow in each case in the direction of the inner face and which open into a free opening edge (“overhang”). This rough inner surface prevents shifts of the content of the shell, so that it is possible to dispense with securing by means of adhesion points. Accordingly, the smooth outer surface of the shell material can be formed by convex material sections extending between the perforations. In contrast to a material that is flat on both sides, such a shell material can be referred to as “three dimensional.”

Here, it is particularly preferable to provide that said three-dimensional shell material is laminated onto the above-mentioned polypropylene nonwoven web. Such a design results in improved fluid uptake properties.

Similarly, it is possible for the wound care article to comprise a fluid-impermeable underwear protection (“backsheet”) on its side facing away from the wound.

It is particularly preferable for the absorbent body to have, in a top view onto its flat side, a face (F1) which in its non crosslinked state is 3% to 75% smaller than the face (F2) of the flatly positioned shell and is freely movable or secured in the shell, wherein the shell has pores over its entire surface, which in each case are smaller than the non crosslinked superabsorbent polymers.

It is preferable to provide that this area ratio is 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74 and/or 75%.

In this manner, it is ensured that the material section, in the case of fluid absorption, can increase in volume and is not restricted by the shell.

It is particularly preferable for the shell to comprise, in a top view onto its flat side, a peripheral excess projecting outward over the seam, and for the absorbent body to have no hard, sharp edges and corners.

In a preferred embodiment, the hygienic or care article according to the invention is a wound care article. The latter comprises copper or copper ions in the form of copper oxide with a minimum particle size of ≧1 μm, which is arranged, together with the superabsorbent polymers, in the material section. The material section comprises at least one shell on its side facing the wound. The pores of this shell have sizes of ≦1 μm.

Since most bacteria have a diameter between approximately 0.1 and 700 μm, this wound care article has the advantage that the bacteria are capable of passing through the shell, wherein the copper oxide can deploy its antimicrobial, that is to say bactericidal, action, and the SAPs bind the bacteria or the toxins released by said bacteria. The copper oxide itself cannot enter the wound, so that it is not absorbed by the body. Although the absorption of copper via a wound, as mentioned above, is in general safe, the wound dressing that has just been described, with which no absorption can take place, is nevertheless accepted more by patients.

In a preferred embodiment, the shell encloses the material section made of superabsorbent polymers of the hygienic or care article according to the invention.

Moreover, on its face facing away from the wound, the shell can be designed to be impermeable to fluids.

In an additional embodiment, the hygienic or care article according to the invention comprises

a) a section comprising soft foam, and/or

b) a wound contact layer, and/or

c) a wound draining device for use with negative pressure.

The section made of soft foam can be at least one material selected from the group containing thermoplastic soft foams, such as polyurethane, polyamide or polyether foam, silicone foam as well as cellulose foam or natural sponge.

The term “wound contact layer” denotes a mesh or gauze like structure which is often positioned on a wound as a so-called “primary bandage” before a secondary bandage is optionally positioned. Wound contact layers are often also referred to as “wound gauzes” and they are used primarily to prevent the entire wound dressing from sticking to the wound. For this purpose, a wound contact layer is manufactured from a material or coated with a material such that adhesion to the wound is reduced or prevented. Alternatively, the geometry of the wound contact layer can be designed so that adhesion to the wound is reduced or prevented.

It is preferable to provide that the primary bandage consists of a silicone or of a silicone coated material or to comprise said material. Thus, for example, it is possible to provide a mesh which consists of a silicone or a silicone coated material or which comprises said material, or a perforated film or a film provided with pores, a perforated or pore forming nonwoven web or woven fabric, or a mesh made of a plastic material—for example, polyethylene, polypropylene or polyamide—which is coated with silicone on at least one side.

The silicone can be structured in such a manner that it has a decidedly adhesive effect, so that the primary bandage adheres to the wound, but at the same time prevents adhesion of the secondary bandage, which may have to be replaced frequently, to the wound, or even its incorporation in granulation tissue. The adhesion properties of silicone can be adjusted technically with great precision, so that a secure adhesion can be ensured, without detachment of the primary bandage causing pain—for example, due to adhering body hair, or even trauma.

Furthermore, a preferably open-celled copper- or copper-ion-containing foam provided with superabsorbent polymers can also be provided. Such a foam can be used, for example, in a negative-pressure device.

Moreover, the hygienic or care article according to the invention can also be introduced in a wound supply system for wound drainage using negative pressure. Such systems are disclosed, for example, in the patents DE202004017052, WO2006048246 and DE202004018245 of the applicant of the present invention, the disclosure content of the mentioned patents are herein incorporated by reference in their entireties.

The grammage of the hygienic or care article according to the invention can be in the range between ≧50 and ≦2000 g/m². Here, it is preferable to use grammages of 50, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1550, 1600, 1650, 1700, 1750, 1800, 1850, 1900, 1950 and/or 2000, in each case ±25 g/m².

The thickness can be in the range between ≧1 and ≦50 mm. It is preferable here to use thicknesses of 2, 4, 6, 8, 10, 12, 14, 16, 18, 20, 22, 24, 26, 28, 30, 32, 34, 36, 38, 40, 42, 44, 46, 48 and/or 50, in each case ±1 mm.

The absorption capacity here can be in the range between ≧3 and ≦5000 mL 0.9% sodium chloride/m² at 0.2 psi. Here, the following values are preferable: 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 0.9% sodium chloride/m².

Alternatively, the absorption capacity here can be in the range between ≧2 and ≦100 g water/g. Here, the following values are preferable: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100 g water/g.

The total content of superabsorbent polymers here can be in the range between ≧5 and ≦100% w/w. Here, the following values are preferable: 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 and/or 100% w/w.

The tensile strength can be in the range between ≧5 and ≦80 N/5 cm. Here, the following values are preferable: 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and/or 80 N/5 cm.

The extensibility here can be in the range between ≧3 and ≦80%. Here, the following values are preferable: 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79 and/or 80%.

In addition, the hygienic or care article according to the invention, or the absorbent body located therein, can comprise repeating patterns or textures, such as a checkers, a punched pattern or the like, for example.

Furthermore, it is particularly preferable to provide that an absorbent body located in said hygienic or care article according to the invention comprises, in addition to a layer comprising hydroactive polymers, at least one second flanking layer, which comprises fewer or no hydroactive polymers and whose surface protrudes beyond the first mentioned layer. In this manner, it is ensured that the layer comprising hydroactive polymers can increase in volume in accordance with the fluid absorption, without the volume increase being detectable from the outside, because the latter layer is concealed by the second layer.

Furthermore, the hygienic or care article according to the invention preferably comprises a shell which in turn comprises means that are designed and/or selected in such a manner that the shell is deformable in a targeted manner at least partially by a volume increase of the absorbent body, caused by the fluid absorption. In principle, such a behavior can be achieved with a unidirectional resilient material, that is to say a material which is extensible in one direction, but not in the direction orthogonal to said first direction.

Furthermore, it is preferable to provide that the hygienic or care article according to the invention comprises a composition containing at least one nutrient, at least one disinfecting or decontaminating and/or at least one protease-inhibiting active substance and/or active substance complex for the external supply and/or for the treatment of wounds of the human or animal body.

Furthermore, the use of a hygienic or care article according to the invention is provided for treating chronic wounds, acute bleeding wounds, burn wounds and traumatically produced wounds.

The term “chronic wounds” denotes wounds that are not primarily due to traumatic actions. Although traumatic actions may have been the original inducing agents of such a wound, the chronic wound is characterized above all by delayed wound healing. Chronic wounds often present only minor bleeding, if any at all, but often produce strong exudation.

The term “minor bleeding” denotes bleeding that is of arterial origin, or possibly venous origin, or interstitial or capillary origin, in each case with an outcome that is not indirectly or directly life threatening.

The term “acute bleeding wounds” denotes wounds that lead to large blood losses. As a rule, arterial bleeding, caused by traumatic actions, for example, is responsible for such wounds. Acute bleeding wounds can under some circumstances be indirectly or directly life threatening. Hemostasis therefore is a very high priority in the case of acute bleeding wounds.

Similarly, the use of a hygienic or care article according to the invention is provided for operative or postoperative care or for military purposes.

Moreover, the use of a hygienic or care article according to one of the previous claims is provided as a patient undersheet and/or as an incontinence sheet. Here the above-mentioned advantages associated with the release of active copper apply particularly if the pH is slightly lowered. The term “patient undersheet or incontinence sheet” denotes products that are used in the care or hospital sector as undersheets for patients or persons in need of care and used to absorb exiting fluids.

Here, it is possible to provide in particular for such a patient undersheet or incontinence sheet to have a substantially flat shape, wherein preferably at at least one of its ends a pouch in the form of a material foldover is formed, into which the feet of a patient or a person in need of care can be introduced. Alternatively, such a pouch can also be used for the disposal of the used undersheet (by wrapping the entire undersheet after use in said pouch), or for receiving hygiene products, such as urine bags, diapers and the like, for example.

This pouch can be attached, for example, by adhesive or welding seams formed in a marginal position.

Moreover, it is preferable for the bottom side of such patient undersheets and/or incontinence sheets to be made of an actively breathing material which is resistant to rupturing and watertight but at the same time allows the passage of air and optionally also water vapor. Textile materials having such properties (produced by a microporous structure, for example) are known on the market, for example, in the wound care sector or the sector of functional textile materials in sportswear.

Said patient undersheet and/or incontinence sheet can, as a result of its being doped with copper or copper ions, also
prevent the development of inflammatory bedsores
contribute to degrading a bacterial biofilm and/or
inhibit the formation of odors.

In all three cases, a synergism with the mentioned superabsorbent polymers comes into play. In the first case, the latter super absorbent polymers absorb the exudates and/or body excretions and thus prevent maceration at the wound margins; in the second case, the latter superabsorbent polymers dry out the biofilm, so that cracks form through which the copper or copper ions can deploy their biofilm-degrading action; and, in the last case, the fluid absorbed is disinfected, so that no odors can form.

Moreover, the use of a hygienic or care article according to the invention as a compression bandage is provided. Such bandages are used, for example, in venous compression therapy, in which local pressure onto the venous vascular system of the legs leads to an increase in the flow rate of the blood. This pressure can be generated by bandaging the leg with compression bandages or by means of special stockings Compression therapy is used for various disease pictures, for example, such as chronic venous insufficiency, post-thrombotic syndrome, primary and secondary lymphedema, primary and secondary varicosis, thrombophlebitis, etc. Many of these indications include, besides the need to absorb fluids (for example, edematous interstitial fluid), also a high risk of infection. Here, the combination of copper or copper ions and superabsorbent polymers represents a considerable potential.

Moreover, a kit for acute, emergency or military medicine care or chronic care is provided, which comprises a hygienic or care article according to the invention.

In addition, according to the invention, a method is provided for treating wounds, in particular chronic wounds, comprising the use of

a) hydroactive polymers, in particular superabsorbent polymers, and

b) a content of copper or copper ions.

The disclosure content of the any of the aforementioned patents is herein incorporated by reference in their entireties.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A hygienic or care article, comprising:
a) a content of hydroactive polymers, and
b) a content of copper or copper ions.

2. The hygienic or care article according to claim 1, wherein the copper ions are selected from the group consisting essentially of copper(I) and copper(II) ions.

3. The hygienic or care article according to claim 1, wherein the copper ions are bound copper ions.

4. The hygienic or care article according to claim 1, wherein the copper oxide comprises particles having a size between ≧0.5 μm and ≦20 μm.

5. The hygienic or care article according to claim 4, wherein the copper or copper ions are present in powder form, granulate form, in wire form, as a colloid, as a fiber impregnation and/or as cuttings.

6. The hygienic or care article according to claim 1, wherein the hydroactive polymers are superabsorbent polymers.

7. The hygienic or care article according to claim 1, further comprising a material section comprising superabsorbent polymers and a shell arranged around in at least some sections of the material section.

8. The hygienic or care article according to claim 1, wherein the hygiene and/or care article comprises:
a) a section comprising soft foam,
b) a wound contact layer, and
c) a wound drainage device for use with negative pressure.

9. Use of a hygienic or care article according to claim 1 for treating chronic wounds, acute bleeding wounds, burn wounds and/or traumatically produced wounds.

10. Use of a hygienic or care article according to claim 1 as a patient undersheet or an incontinence sheet.

11. Use of a hygienic or care article according to claim 1 as a compression bandage.

12. A method for treating wounds, in particular chronic wounds, comprising:
a) using a plurality of hydroactive polymer including superabsorbent polymers, and
b) using a content of copper or copper ions.
